(19) United States
(12) Patent Application Publication
(10) Pub. No.: US 2003/0225327 A1
(43) Pub. Date: Dec. 4, 2003

(54) Compressible foot plate to monitor pressure

(76) Inventors: Jan A.G. Willen, Krikonvagen (SE); Arne Gaulitz, Bolldals Aspvagen (SE); Barbro Danielsson, Almvagen (SE); Thomas Nicklasson, Kuttervagen (SE)

Correspondence Address: Samuels, Gauthier & Stevens LLP, Suite 3300, 225 Franklin Street, Boston, MA, 02110, US

(21) Appl. No.: 10/435,727

(22) Filed: May 8, 2003

Related U.S. Application Data
(63) Continuation of application No. PCT/SE01/02502, filed on Nov. 12, 2001.

(30) Foreign Application Priority Data
Nov. 13, 2000 (SE) 0004130-1

(51) Int. Cl. A61B 5/05; A61B 6/00

(52) U.S. Cl. 600/415; 378/209; 378/20

(57) ABSTRACT

A compressible footplate for a device for complementary examination of the lumbar region at computer tomography and magnetic resonance tomography, whereby it comprises a resting plane being part of a patient table, a footsupport being arranged to the resting plane comprising at least one compressible footplate. The compressible footplate comprises a footplate, a compressible means, an indication means and a holder-on, preferable in the form of an outer house.

### DESCRIPTION

1. Technical Field

The present invention relates to a device comprising a compressible footplate having an indicator means and a display unit suitable to be used in a device for complementary examination of the lumbar region at computer tomography and magnetic resonance tomography.

The object of the present invention is to obtain a footplate for monitoring pressure, the indication of said footplate being independent from of where and how the pressure is applied, which footplate is suitable to be used in a device with the help of which a careful and reproducible examination of the lumbar region in connection with computer tomography and magnetic resonance tomography can be carried out, in particular for the diagnosis of changes of the spinal cord and its nerve structures (spinal stenosis).

### BACKGROUND OF THE INVENTION

Traditionally, patients have been examined in a relaxed position on their backs at computer tomography and magnetic resonance tomography, which means that structures in connection with the spinal cord channel, which are variable, e.g., intervertebral discs, ligaments, and small joints, are subject to a minimal load. At load i.a., the pressure in the intervertebral disc will increase, which results in a protuberance, which in turn reduces the space in the spinal cord channel where nerve and vessel structures available are present. At the examination of the spinal cord channel of the lumbar region using computer tomography or magnetic resonance tomography suspecting spinal stenosis there is a demand for being able to load the lumbar region in the same way as takes place when standing right up, simultaneously as the patient is placed in horizontal position.

In U.S. Pat. No. 3,629,581 a device is disclosed for positioning a patient's shoulders in connection with an X-ray examination of a patient's spine, whereby the upper vertebral column is pressed down towards the examination table and facilitates a good X-ray imaging of the upper vertebras. Hereby a pressure is applied over the vertebral column of the lying patient by means of two flexible traction lines provided with handles for the patient, which traction lines are arranged around a footplate.

In EP-A-0 714 265 a device for the compression of the lumbar region for medicinal imaging is disclosed, comprising a resting plane to which a foot support is applied containing pressure sensors arranged to register the pressure from the respective foot on said footplate, a waistcoat arranged to be placed around the body of a patient, traction lines being arranged between said footplate and said waistcoat, as well as a straining device to said traction lines, which device is arranged to be strained to a predetermined value.

The design of the footplate can be directly connected to lack of carefulness of the determination result, whereby there is a problem present at the design of a compressible footplate which indicates a pressure thereon independent of where and how the pressure is applied onto the footplate, which can used in a device which can be utilised at the examination of the spinal cord channel of the lumbar region.

2. Description of the Present Invention

The present invention encompasses a pressure footplate suitable for reproducible indication in a device for the compression of the lumbar region for the medical imaging. Besides waistcoat, traction lines and a resting plane a footplate is to be included which to a considerably degree contributes to the carefulness of said device for the compression of the lumbar region.

The compressible footplate comprises a footplate, a compressible device, an indicator means, and an outer house and the Cupertino thembetween, i.e., both the constructions of the same, and wherein the fitting and the mounting of the compressible footplate determine the carefulness of the final determination result.

### DETAILED DESCRIPTION OF THE INVENTION

The present invention relates to a compressible footplate of a device used at complementary examination of the lumbar region at computer tomography, whereby it comprises a resting plane being part of a patient table, a foot support arranged to said resting plane said support comprising at least one compressible footplate, whereby the compressible footplate comprises a footplate, a compressible device, an indicator means and a contra support, preferably in the form of an outer house, which is characterized in that the compressible device comprises at least four plates having a higher hardness and at least two plates having lower hardness than the first mentioned plates, and at least two distance means, whereby said plates and distance means are arranged in such a way in relation to each other that the two parallel plates having a lower hardness contributes to said device being compressible.

According to a preferred embodiment of the invention the distance means have sides provide with semi-circular recesses with a substantially circular or oval cross-section or the similar, which are turned inward towards each other in a mounted position of the compressible device in order to create better compression properties and a higher resistance.

According to another preferred embodiment of the invention an indicator means comprises at least two rolls, an adjustable slide, an elastic band, and a wire, which are arranged to counteract each other, a pointing means, whereby said indicator means is arranged to influence an outer movement/force and to transform the outer movement/force to a value via a display unit.

### DESCRIPTION OF THE FIGURES

FIG. 1a shows a side view of an embodiment of a complete device for compression of the lumbar region for medical imaging and with the footplate seen in its environment.

FIG. 1b shows a side view of an enlargement of the footplate with its ingoing details.

FIG. 2 shows a view of the footplate seen from above.

FIG. 3 shows a side view of the compressible device.

FIG. 4 shows an indicator means seen in a cross-section from above and from the side.

FIG. 5a shows the outer house seen from above.

FIG. 5b shows the outer house seen from below.

### PREFERRED EMBODIMENT

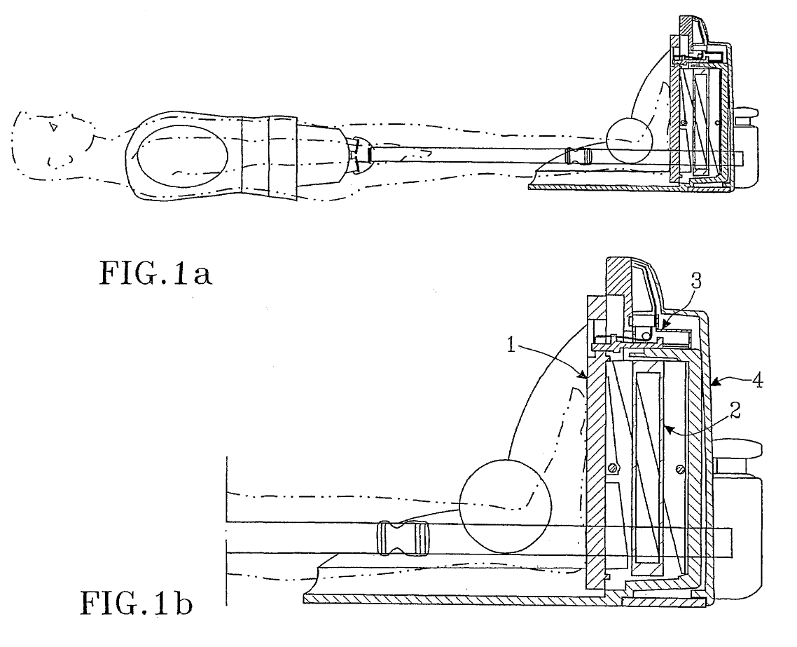

The compressible footplate seen in its environment is a complete device for the compression of the lumbar region for a medical imaging, cf. FIG. 1a.

The device, i.e., the compressible footplate comprises a footplate 1, a compressible means 2, an indication means 3 and an outer house 4, cf. FIG. 1b.

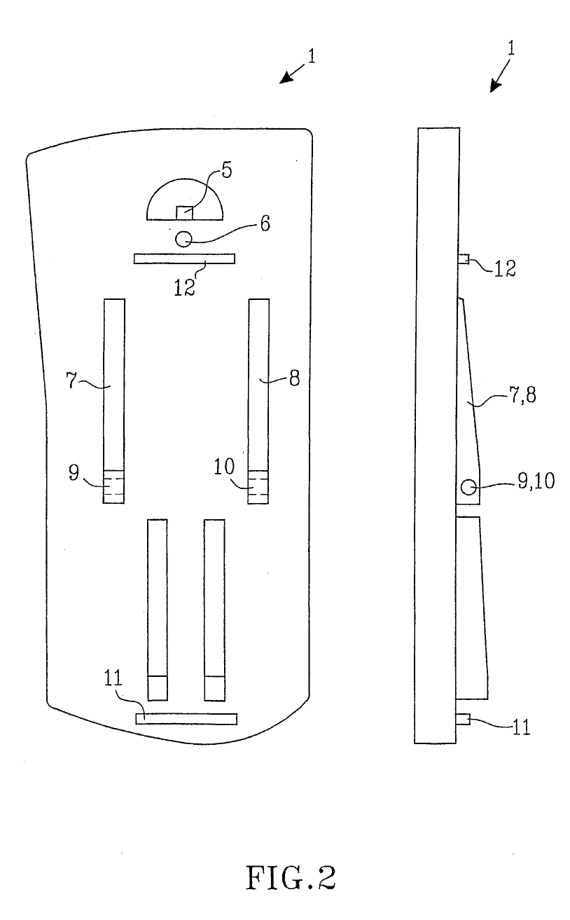

The footplate 1 has a substantially rectangular form having rounded short ends, cf. FIG. 2. The front side of the footplate 1 is a contact surface of a patient's foot. On the rear side of the footplate 1 the compressible means 2 is arranged, which in a mounted condition is covered by the outer house 4 of the device, cf. FIG. 1b.

On the outmost end of one of the rounded short sides of the footplate 1 there is a protrusion 5 and screw hole 6 arranged for an adjustable screw, cf. FIG. 1b, which will be described more in detail in the following.

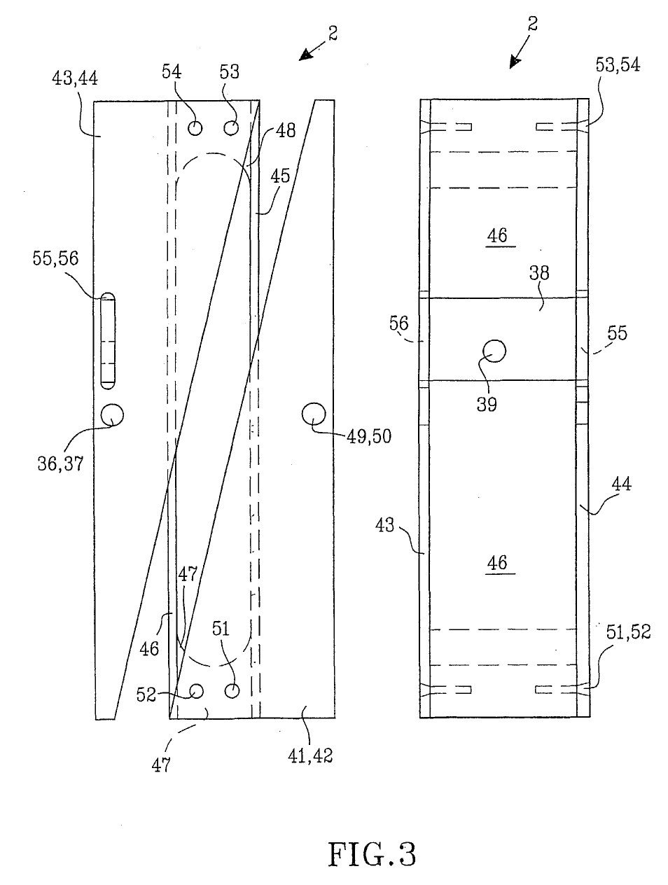

In a preferred embodiment the compressible means 2 encompasses four hard discs 41, 42, 43, 44, two bendable discs 45, 46, two distance means 47, 48, one stability washer 38 and a number of fastening elements, cf. FIG. 3.

Further in a preferred embodiment all four hard discs 41, 42, 43, 44 have a substantially right-angled triangular form and are preferably of the same size. One of the right sides of the triangularly shaped, hard discs, i.e., one cathetus, s about 4 times longer than the other right side, i.e., than the other cathetus. A somewhat convex protuberance is arranged longitudinally along the edges of the right sides of the lower and upper hard discs 41, 42, 43, 44. This creates a better fitting when mounting when the somewhat convex edges will be pressed out rectilinearly when they are fixed to the footplate 1 and the outer house 4.

The two bendable plates 45, 46 have a substantially oblong, rectangular form and are of the same size.

The two distance washers 47, 48 have both a designs as right block. In a particularly preferred embodiment the two distance washers 47, 48 are provided with each their side with a semi circular recess with a circular or oval cross-section or the similar to create a better strength at compression of the compressible means 2, FIG. 3.

At the mounting of the compressible means 2 onto the footplate 1 two 41, 42 of the four hard discs 41, 42, 43, 44 are arranged on their ends having the longer right sides as contact surface with the footplate 1, and are arranged on a distance from each other, and substantially parallel to each other.

A fixation of the two hard discs 41, 42 on the footplate 1 is carried out by means of two protrusions 7, 8. The protrusions 7,8 are arranged on the footplate 1 in such away that they are arranged on either side of the said hard discs 41, 42 when these are arranged on end on the footplate 1. In a lateral view the protrusion 7, 8 have a substantially triangular, right-angled form.

In the highest points of said protrusions 7, 8 there are through-going holes 9, 10 being arranged. The two hard discs 41, 42 are arranged with fitting holes 49, 50 for the arrangement of a through-going fixation joint, i.e., to fixedly secure the two said hard discs 41, 42 onto said footplate 1.

The compressible means 2 further comprises two distance washer 47, 48. The first one 47 is arranged by means of a joint between two of the vertical side surfaces of the distance 47 to the two pointed upper, inner situated corners of the two first hard discs 41, 42, when these are arranged on end, cf. FIG. 3.

On the underneath side of the distance 47 there is a surface of an end of the first bendable plate 45 being arranged, which then is situated above the first bendable plate 45 and the footplate 1, and is substantially parallel to both the first bendable plate 45 and the footplate 1. A second distance 48 is arranged vertically in a corresponding manner by means of a joint between the upper and lower sides respectively, of opposite ends of the two bendable plates 45, 46, cf. FIG. 3. The third and forth hard discs 43, 44 are arranged on end, however, in such away that they are reversed in relation to the first and second hard discs 41, 42. The vertical side surfaces of the distance 48 are joined to the pointed lower, inner corners of the hard discs 43, 44 when they are arranged on end in accordance with above, cf. FIG. 3.

A stabilization washer 38 is arranged in an oblong rounded through-going hole 55,56 in the upper part of each of the two upper hard discs 43, 44 to create further stability of the compressible means 2. In the centre of the stability washer 38 a screw hole 39 is arranged for a fixed mounting to the outer house 4 at the mounting thereof, which will be described more in detail in the following.

Hard discs, bendable discs and distances are preferably fixedly arranged and joined to each other by means of an adhesive which is then temperature hardened. The edges between distance and hard disc, and between distance and bendable disc, respectively, are bevelled for the purpose of obtaining a better strength when sharp fillet joints are avoided. The sides of the distances 47, 48 having semi-circular recesses are turned inward towards each other in a mounted position of the compressible means 2 to create better compression properties and to obtain a better strength, cf. FIG. 3.

In two through-going holes 51, 52, and 53, 54, preferably arranged in the same manner of the two hard discs 41, 42, and 43, 44, respectively, and in accordance with the holes of the respectively distance 47, 48 there is shaft, screw or the similar used as a further fastening element besides the adhesive.

When the footplate 1 is pressed in a preferred embodiment this leads to that the first bendable plate 45 is bent in such away that it will come closer to the rear side of the footplate 1 at the compression. The second bendable plate 46 is then bent in a similar way so that it will come closer to the bendable plate 45 and the footplate 1. Thus it is substantially the bendable plates 45, 46 which are bent at the compression, which leads to that it is primarily the bendable plates 45, 46, the second distance 48, and the upper two hard discs 43, 44 which are moved out of their starting positions and closer to the rear side of the footplate 1. Accordingly, the third and fourth hard discs 43, 44 are brought closer to the first and second hard, rigid discs 41, 42 at the compression, so that the gap present at a non-compressed condition is reduced during compression.

In a particularly preferred embodiment the four vertical, triangular discs 41, 42, 43, 44 are suitably made of glass fibre reinforced, hardened polymer, and the two horizontal, rectangular plates 45, 46 are preferably made of a carbon fibre reinforced polymer, such as KEVLAR®.

The distances 47, 48 are preferably made of a hardened polymer.

Concerning the footplate 1 it is arranged with two further oblong angular protrusions 11, 12, which are arranged perpendicular to the first protrusions 7, 8. The two shorter protrusions 11, 12 contributes to that a perfect fitting is obtained between the footplate 1 and the outer house 4 of a mounted device, which will be discussed more in detail in the following.

The footplate 1 is preferably made of hardened polymer, such as polyurethane.

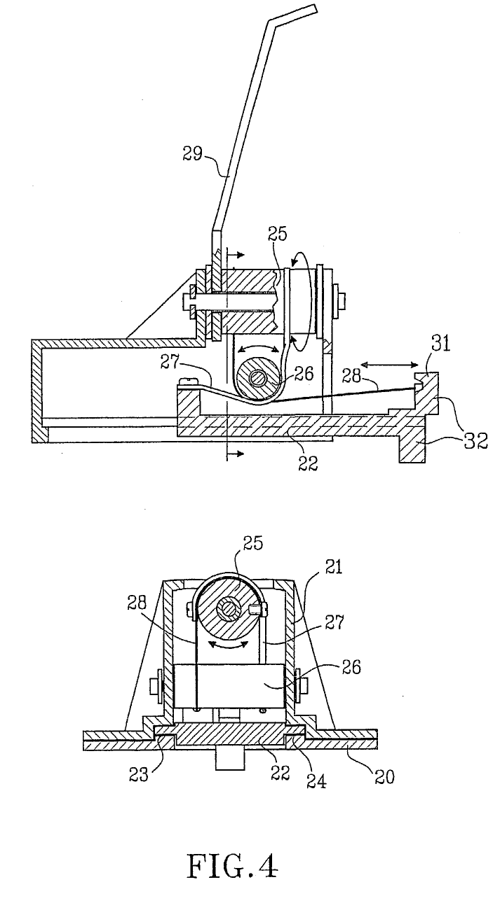

The task of the indication means 3 is to indicate a compression of the compressible means 2 and to transfer that to a readable value. The indication means 3 comprises a bottom plate 20, an upper spherical lid 21, a slidable slide 22, two rotating rolls 25, 26, an elastic band 27, a thread 28, and a pointer 29, cf. FIG. 4. In a preferred embodiment the bottom plate 20 abuts a side surface of the outer house 4 and the upper spherical lid 21 is arranged on top of the bottom plate 20. A slide 22, preferably in the form of a disc can slide along two tracks 23, 24, which are formed inside the indication means 3 between the bottom plate 20 and the upper spherical lid 21, and perpendicular to a surface 58 where a display unit 59 is arranged. On one of the edges of the slide 22 there is a protrusion 31, as well, having a smooth side surface 32 arranged perpendicular to the sliding direction of the slide 22, cf. FIG. 4.

The indication means 3 comprises in a mounted condition the two rolls 25, 26, as well. The first roll 25 is made rotating around its axis and is arranged parallel to the slidable slide 22, and perpendicular to the surface 58 of said display unit 59. The second roll 26 is rotating around its axis, as well, and is arranged perpendicular to the first roll 25, and parallel to the slidable slide 22, cf. FIG. 4. Both rolls 25, 26 are rotating around their axis in both directions within the indication means 3.

In a preferred embodiment a first end of an elastic band 27 is fixedly arranged in the end of the slide 22 facing the protrusion 31, cf. FIG. 4. A second end of the elastic band 27 is fixedly arranged to the outer surface of the first roll 25 with an extension via the second roll 26. When both rolls 25, 26 are rotating the elastic band 27 slides from the first roll 25, via the second roll 26, and towards the end position of the slidable slide 22, and back to its starting position after compression.

The said elastic band consists, preferably, of a silicon rubber or the similar. Further, in said embodiment a first end of a thread 28, substantially without elasticity, is fixedly arranged with its one end to a part of the smooth surface 32 of the slide and with a second end fixedly arranged to the outer surface of the first roll 25 with an extension via the second roll 26. When the two rolls are rotating the thread 28 slides from the first roll 25, via the second roll 26, and to the second end position at the protrusion 31 of slidable slide 22 and back to its starting position after compression.

Both the elastic band 27 and the thread 28 are fixedly arranged to the envelope surface of the first roll 25. They are, however, arranged in substantially opposite side to each other and in opposite directions to each other over the first 25 and second roll 26. At a compression of the footplate 1 this leads to that the elastic band 27 and the thread 28 contracts each other.

In a preferred embodiment one of the sliding directions of the slide 22 is restricted by said protrusion 31, and its second sliding direction is restricted by the smooth surface 32 of the slide 22 when the device has been mounted.

Further the indication means 3 comprises a pointer 29 which is fixedly arranged to the first roll 25 and is preferably fixedly surrounding its envelope surface. The pointer 29 is arranged parallel to and in close vicinity to the surface of the display unit in order to avoid parallax effect.

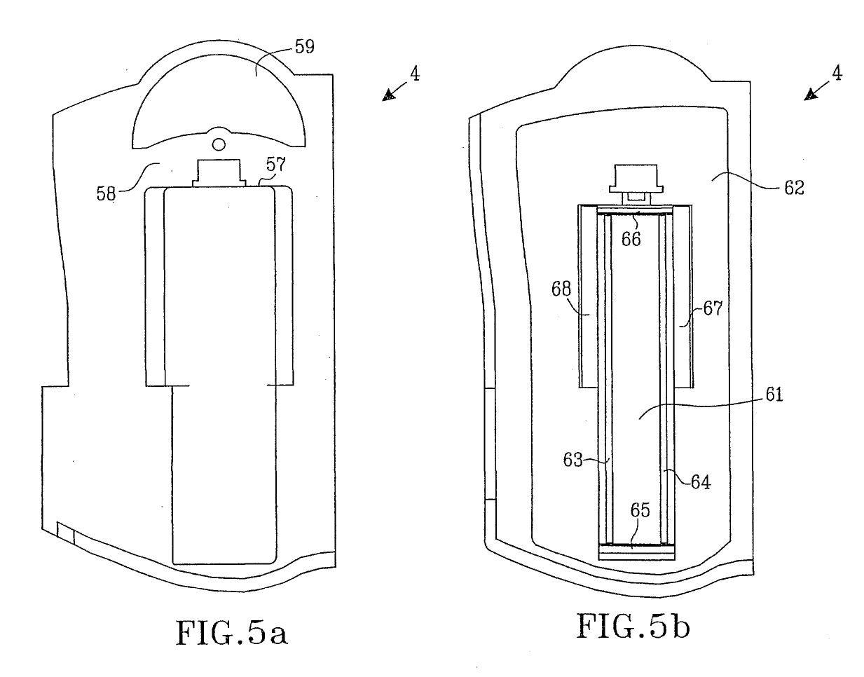

In a particularly preferred embodiment rotation of the roll 25 around its axis within the indication means 3 leads to a deflection of the pointer 29. The deflection can be read on the display unit 59, when the angle, as the pointer 29 moves, when the first roll 25 rotates corresponds to one scale deflection, cf. FIGS. 4 and 5b. The pointer can also be connected to a monitoring unit for monitoring the value obtained. The components of the indication means are preferably made of hardened polymer.

The are also other embodiments of the indication means, which then can be applied in a different way in relation to the display units and pointers.

The outer surrounding house 4 of the device for compression of the lumbar region is arranged with a space 61 for applying the compressible means 2 and has a recess 62 for receiving the footplate 1, cf. FIG. 5b.

The space 61 is arranged with tracks 63, 64 for receiving the edges of the upper hard discs 43, 44 of the compressible device 4 and with tracks 65, 66 for the said shorter protrusions 11, 12 of the footplate 1. All said tracks of the outer house 4 creates a substantially perfect fitting in a mounted condition of the device, which thereby leads to that the device for compression of the lumbar region carries out very careful measurements.

In connection to the space 61 and the recess 62 two further tracks 67, 68 are arranged to be fit in with the two protrusions 7, 8 of the footplate 1.

There is a suitable clearance between the outer side edges of the footplate 1, and its rear side and the inner edges of the space 61 and the recess 62 of the outer house 4 so that these parts can move freely at a compression of the footplate 1 and the compressible means 2. In a preferred embodiment a distance remains of about 4 mm between the footplate 1 and the surface of the recess 62 of the outer house 4 at a complete compression.

There are other embodiments of the outer house 4, which, however, preferably has a function as a counter holder-on to the footplate 1 and the compressible means 2. Fixation between the footplate 1 and the compressible means 2 has been discussed previously. The outer house 4 and the compressible means 2 are, in turn, fixedly arranged by means of fastening elements through a through-going screw hole of the lower space 61 of the outer house 4 and through a screw hole 39 of the stability washer 38 of the compressible means 2. There is then a good fitting between the upper hard discs 43, 44 and the inner tracks 63, 64, and the protrusions 11, 12 of the footplate 1 and the tracks 65, 66 of the outer house 4.

On a side surface 57 of the outer house 4 the indication means 3 is arranged by means of fastening element and is arranged perpendicular to a surface 58 where a display unit 59 is applied, which is previously mentioned. A suitable gap can be arranged between the inner side of the spherical lid 21 and the surface 58, as well, where the display unit 59 is arranged.

A hole is furthermore arranged in the outer house 4 for the movement of the slidable slide 22 and the protrusion 31, and to create a firm abutment between the protrusion 31 of the slide 22 and the protrusion 5 of the footplate 1.

The said protrusions 31, 5 are surrounded by an O-ring at the mounting for a substantially exact abutment surface without any clearance between the footplate 1, the compression means 2, the indication means 3 and the outer house 4, which is required for a careful calibration.

The material of the outer house 4 is preferably hardened polymer. The O-ring is preferably of an elastic rubber material.

In a particularly preferred embodiment adjustment and calibration are carried out by means of an adjustment screw, which is arranged in the screw hole 6 of the footplate 1, as previously mentioned. The adjustment screw is arranged with its point against the said smooth surface 32 of the slidable slide 22. When the pointer 29 is adjusted to a zero-position on the display unit 59 the point of the adjustment screw is brought into contact with the smooth surface 32 of the slide 22, however, without forcing this out of its position. The smooth surface 32 then functions as a calibration and stop surface.

The function of the compressible footplate will now be described more in detail. When the footplate 1 is compressed and the compressible means subsequently is compressed, then the adjustment screw of the footplate 1 is pressed against the smooth surface 32 of the slide 22. Then the slidable slide 22 is pressed further into the tracks 23, 24. The slide 22 forces by means of the elastic band 27 the first roll 25 via the second roll 26. Then the first roll 25 rotates a certain angle around its axis whereby the pointer 29 provides an angle deflection, which can be read on the display unit 59.

When a patient's foot still no longer compresses the footplate 1 the O-ring contributes to returning the slide 22 to its starting position. The thread co-operates with the second roll 26, when it slides and drives thereby the first roll 25, which rotates to its starting position, whereby the pointer 29 is returned to its starting position.

One may also consider embodiments of the invention both with regard to the footplate 1, the compressible means 2, the indication means 3 and the outer house 4.

Finally, it is noted that the invention can be utilized for other compressible discs in connection with other applications.

1. Compressible footplate for a device for complementary examination of the lumbar region at computer tomography and magnetic resonance tomography, whereby it comprises a resting plane being part of a patient table, a footsupport being arranged to the resting plane comprising at least one compressible footplate (1), whereby the compressible footplate comprises a footplate (1), a compressible means (2), an indication means (3) and a holder-on, preferably in the form of an outer house (4),
characterized in
that the compressible device comprises at least four plates having a higher hardness (41, 42, 43, 44) and at least two plates (45, 46) having lower hardness than the first mentioned plates, and at least two distance means (47, 48), whereby said plates (41, 42, 43, 44, 45, 46) and distance means (47, 48) are arranged in such a way in relation to each other that two parallel plates (45, 46) having a lower hardness contributes to said device being compressible.

2. Device according to claim 1,
characterized in
that the distance means (47, 48) have sides provide with semi-circular recesses with a substantially circular or oval cross-section or the similar, which are turned inward towards each other in a mounted position of the compressible means (2) in order to create better compression properties and a higher resistance.

3. Device according to claim 1,
characterized in
that an indicator means (3) comprises at least two rolls (25, 26), an adjustable slide (22), an elastic band (27), and a wire (28), which are arranged to counteract each other, a pointing means (29), whereby said indicator means (3) is arranged to influence an outer movement/force and to transform the outer movement/force to a value via a display unit (59).
